(19) United States
(12) Patent Application Publication
(10) Pub. No.: US 2026/0351421 A1
(43) Pub. Date: Nov. 5, 2026

(54) Methods for diagnosing and treating colorectal cancer

(71) Applicant: Sanford Health, Sioux Falls, SD (US)

(72) Inventor: Max Weaver, Sioux Falls, SD (US)

(21) Appl. No.: 19/666,251

(22) Filed: May 2, 2026

Related U.S. Application Data
(60) Provisional application No. 63/799,000, filed on May 2, 2025.

(51) Int. Cl. G16H 50/30; G06N 20/20; G16H 10/60; G16H 20/40; G16H 50/20; G16H 50/70

(52) U.S. Cl. CPC G16H 50/30; G06N 20/20; G16H 10/60; G16H 20/40; G16H 50/20; G16H 50/70

(57) ABSTRACT

Improved methods are provided for training and executing models to predict whether a patent is at elevated risk of colorectal cancer and thus should be assessed via colonoscopy, stool sample, or other diagnostic technique to detect the current or likely future presence of colorectal cancer in a patient. These techniques reduce the computational cost of evaluating large numbers of widely available but, per conventional wisdom, unrelated variables that are often present in a patient's medical record or otherwise accessible at low cost (e.g., without performing expensive diagnostics). This allows patients who do not exhibit any of the ‘conventional’ indicia of colorectal cancer risk (e.g., family or personal history of polyps, cancer, etc.) but who exhibit increased risk of colorectal cancer to be targeted for colonoscopy, stool sample evaluation, or other diagnostics.

### CROSS-REFERENCE TO RELATED APPLICATION

The present application is a non-provisional patent application claiming priority to U.S. Provisional Patent Application No. 63/799,000, filed on May 2, 2025, the contents of which are hereby incorporated by reference.

### BACKGROUND

Colorectal cancer causes significant death and disability annually. Early detection can significantly reduce morbidity and mortality. However, methods for early detection, like colonoscopies or even testing of stool samples, can be expensive and invasive. Additionally, patient compliance with recommended testing schedules can be poor. While certain canonical indicia (e.g., a personal or family history of colorectal cancer or related colorectal symptoms) are used as strong indicators that further and/or enhanced screening is indicated. However, modern electronic health record systems provide large amounts of data that could be investigated for utility in identifying, at low cost, additional patients to subject to further and/or enhanced screening and, in so doing, reduce overall morbidity and mortality related to colorectal cancer.

### SUMMARY

In a first aspect, a method for diagnosing a patient as at an increased risk of colorectal cancer relative to control is provided that includes: (i) obtaining, for a patient, respective diagnostic measurements for each feature of a set of input features; (ii) applying the diagnostic measurements for the patient to a trained machine learning model to generate, for the patient, a risk of colorectal cancer, wherein the trained machine learning model has been trained via a training process that includes: (a) obtaining a training dataset that comprises, for each subject of a plurality of subjects, a corresponding set of diagnostic measurements for each feature of a set of candidate features and a corresponding observed risk of colorectal cancer; (b) based on the training dataset, determining a first subset of the candidate features that are more predictive of the observed risk of colorectal cancer with respect to a bivariate correlative statistic; (c) determining a second subset of features of the first subset that are more predictive of the observed risk of colorectal cancer based on training respective predictive models that omit the respective features of the first subset, wherein the second subset of features is the set of input features; and (d) training the trained machine learning model to predict risk of colorectal cancer using the second subset of features; (iii) determining that the risk is greater than a threshold risk; and (iv) responsive to determining that the risk is greater than the threshold risk, displaying an instruction to perform a colorectal cancer diagnostic test on the patient.

In another aspect an article of manufacture is provided that includes a non-transitory computer-readable medium, having stored therein instructions executable by a computing device to cause the computing device to perform the above method.

In another aspect a system is provided that includes: (i) one or more processors; and (ii) a non-transitory computer-readable medium, having stored therein instructions executable by the one or more processors to cause the system to perform the above method.

These as well as other aspects, advantages, and alternatives will become apparent to those of ordinary skill in the art by reading the following detailed description with reference where appropriate to the accompanying drawings. Further, it should be understood that the description provided in this summary section and elsewhere in this document is intended to illustrate the claimed subject matter by way of example and not by way of limitation.

### BRIEF DESCRIPTION OF THE FIGURES

FIG. 1 is a flowchart illustrating aspects of a method for patient stratification and treatment, according to an example embodiment.

FIG. 2 is a flowchart illustrating aspects of a method for feature selection and model training, according to an example embodiment.

FIG. 3 illustrates experimental results.

FIG. 4 illustrates an example method.

FIG. 5 is a diagram illustrating training and inference phases of a machine learning model, in accordance with example embodiments.

FIG. 6 is a simplified block diagram showing some of the components of an example computing system.

### DETAILED DESCRIPTION

Examples of methods and systems are described herein. It should be understood that the words “exemplary,” “example,” and “illustrative,” are used herein to mean “serving as an example, instance, or illustration.” Any embodiment or feature described herein as “exemplary,” “example,” or “illustrative,” is not necessarily to be construed as preferred or advantageous over other embodiments or features. Further, the exemplary embodiments described herein are not meant to be limiting. It will be readily understood that certain aspects of the disclosed systems and methods can be arranged and combined in a wide variety of different configurations.

As used herein, the singular forms “a”, “an” and “the” include plural referents unless the context clearly dictates otherwise. “And” as used herein is interchangeably used with “or” unless expressly stated otherwise.

All embodiments of any aspect of the disclosure can be used in combination, unless the context clearly dictates otherwise.

### I. OVERVIEW

Colorectal cancer causes significant morbidity and mortality annually. Fortunately, colonoscopies, stool sample testing, or other diagnostic techniques can, if performed early enough in the development of such a cancer, lead to significantly improved outcomes with respect to health and mortality. However, these early warning diagnostics implicate significant costs, including the time of healthcare providers, the use of equipment and supplies to provide such interventions, patient comfort and safety (e.g., related to the inherent risk of anesthesia which may accompany a colonoscopy), procedure rooms or other limited healthcare system resources, or other costs. Additionally, compliance with standard screening schedules can be difficult to elicit from the population at large due to discomfort, time and monetary costs, fear, or other factors. Accordingly, it would be very beneficial to be able to target such screenings to higher-risk individuals, preferably based on already-available demographic or other easy-to-access measures, in order to increase the proportion of colorectal cancers that are detected early while reducing the various costs of such detection.

A set of ‘conventional’ indicators has been used to reduce the recommended age of first colonoscopy, stool sample testing, or other colorectal cancer assessment and/or to increase the frequency at which such assessments are repeated. Such ‘conventional indicators can include a personal or familial history of colorectal polyps or colorectal cancer, a personal history of inflammatory bowel disease (e.g., Crohn's, IBS, colitis), or the presence of one of a specified set of inherited syndromes that is correlated with an increased risk of colorectal cancer (e.g., Lynch Syndrome, familial adenomatous polyposis (FAP), attenuated FAP, Gardner syndrome, Turcot syndrome, Peutz-Jeghers syndrome, MUTYH-associated polyposis). However, modern medical care results in the acquisition of significant amounts of medical health data that could be predictive of colorectal cancer risk and that, thus, could be applied at low cost (due to the data already having been collected) to identify additional individuals who are not positive with respect to any of the ‘conventional’ indicators but whose risk of occurrence of colorectal cancer is sufficiently elevated to justify providing them with a colonoscopy or other colorectal cancer diagnostic. A sensitive, accurate predictor operating on such readily-available predictive data could be used to target additional ‘non conventional’ patients at risk of colorectal cancer, allowing large numbers of additional colorectal cancers to be detected without the costs of blanket testing (e.g., reducing the default recommended age of first colonoscopy) or other naïve screening schemes.

However, while large amounts of medical data may be available that can be used to predict colorectal cancer risk (alone, or in combination with the ‘conventional’ factors), it is difficult to identify which of the available features within the data to use to make such predictions. In addition to the risks of statistically over-powering a prediction by including too many weakly predictive features as input, the use of larger input feature sets also results in significantly greater computational costs (e.g., processor cycles, memory, network interconnect bandwidth use, non-volatile storage, power) both to train such predictive models and to execute (or ‘inference’) them, once trained, to generate predictions for specific patients.

The embodiments described herein overcome these limitations, facilitating the generation of accurate, specific predictor models that can identify, using potentially dozens or other numbers of ‘non conventional’ input features, patients who do exhibit increased risk of colorectal cancer without exhibiting any of the ‘conventional’ factors that might trigger early or more frequent screening. These benefits can be obtained without the significant computational cost of prior feature selection method (e.g., a naïve combinatoric search of the set of sets of available features) via a two-stage feature selection process. The first stage employs a computationally inexpensive process to generate, for each feature of a set of available candidate features, a bivariate correlative statistic between a given feature and the observed risk of colorectal cancer. These correlative statistics are then used (e.g., retaining all features with statistics above a threshold value, retaining the top N features, retaining a specified top percentage of the features) to select a subset of the candidate features for evaluation via a more computationally expensive process in the second stage. The second stage evaluation then determines the final input feature set by selecting, from the first-stage features, those features that are more predictive of the observed colorectal cancer risk. By employing such a two-stage feature selection process, the overall cost of identifying a subset of predictive features from a large population of candidate features can be significantly reduced (e.g., relative to performing the more expensive second stage assessment on all of the available features) which still ensuring that the most predictive features are selected (by using the less computationally expensive but still discriminatory test of the first stage to perform a first selection from within the available features).

Aspects of such a two-stage feature selection process are depicted, by way of non-limiting example, in FIG. 2. A training dataset that includes a first set 201a of candidate features for a plurality of different individuals (e.g., patients whose medical records are available to train a model and which include class, value, status, or other information about the candidate features) is subjected to a first stage 210 to identify, within the set of candidate features 201a, a first subset of features 201b that are more predictive of an observed risk of colorectal cancer with respect to respective bivariate correlative statistics determined for each of the candidate features. This could include determining the bivariate correlative statistic for each of the candidate features 201a and then retaining all of the features whose bivariate correlative statistic is greater than a threshold value, retaining the top N features with respect to the bivariate correlative statistic, retaining a specified percentage of the top features with respect to the bivariate correlative statistic, or performing some other partitioning of the candidate features 201a. The bivariate correlative statistic could include a chi-squared test, a Fisher's exact test, a Wilcoxon rank sum test, or some other statistic that represents a degree of correlation or predictive power between a given candidate input feature and the observed risk of colorectal cancer.

The first subset 201b of features is then subjected to a second stage 220 to identify, within the first subset 201b, a second subset of features 201c that are more predictive of the observed risk of colorectal cancer based on training respective predictive models that omit the respective features of the first subset. Such a feature evaluation and selection process is more computationally expensive (e.g., with respect to processor cycles, memory, network interconnect bandwidth, non-volatile storage, power) than the use of bivariate correlative statistics in the first stage 210, since it includes training a respective model for each feature in the first subset 201b to predict colorectal cancer risk without the feature being evaluated (e.g., using all of features of the first subset 201b other than the feature being evaluated). However, such a feature evaluation process is also, as a benefit of this added computational cost, a more accurate assessment of the individual predictive contribution of each feature within the context of the other available features. The two-stage feature selection process described herein results in the identification of highly predictive features within a set of candidate features while incurring less overall computational expense, since the more computationally expensive model-training-based feature evaluation process is reserved for only the subset of features that pass with respect to the less-computationally-expensive bivariate correlative statistic test.

The second stage 220 could include a variety of different methods of quantifying and ranking the absolute or relative predictive power of each of the features in the first subset 201b. For example, a Shapley additive explanation (SHAP) score could be determined for each of the features in the first subset 201b by training, for a given feature, a respective to predict colorectal cancer risk using all of the features in the first subset 201b except the given feature being evaluated. Such scores (SHAP or otherwise) could then be ranked and used to select the second subset of features 201c of the first subset 201b that are more predictive of the observed risk of colorectal cancer. This could include retaining all of the features whose predictive score is greater than a threshold value, retaining the top N features with respect to the predictive score, retaining a specified percentage of the top features with respect to the predictive score, or performing some other partitioning of the candidate features 201b.

Once the second subset 201c is identified, an output (or “production”) predictor model 120 is trained 230 to predict the observed risk of colorectal cancer based on the features of the second subset 230. The predictor model 120 can then be used to generate predicted colorectal cancer risk scores for patient, and those risk scores can be used to determine whether to provide patients with a diagnostic screening (e.g., stool sample analysis, colonoscopy), whether to increase or otherwise change a frequency of such screening, or to perform some other diagnostic or treatment process.

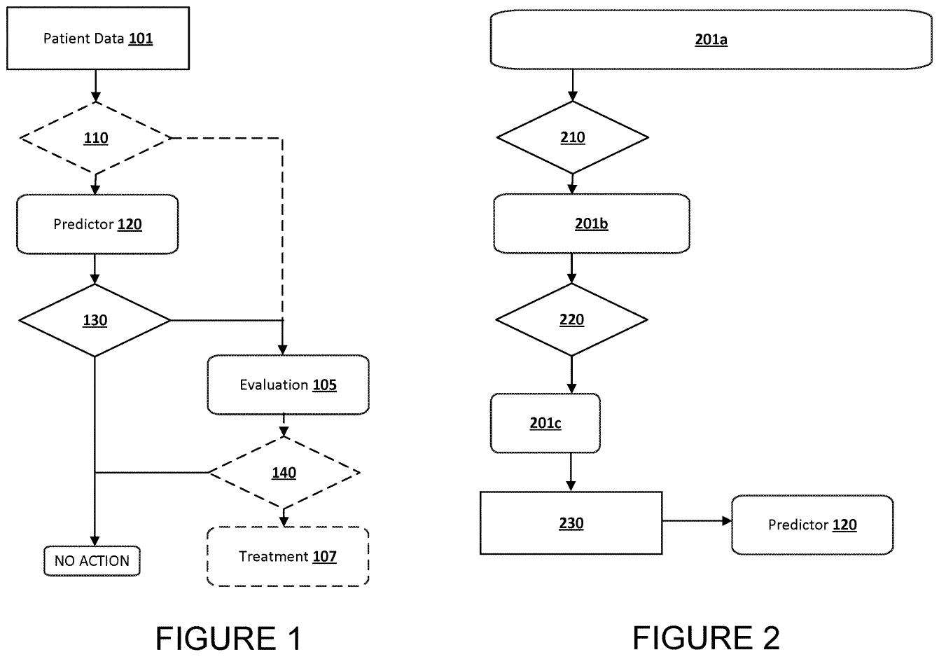

FIG. 1 illustrates the use of the trained predictor model in such a manner. Patient data 101, which includes information for all of the features in the identified input feature set (corresponding to the second subset 201c of FIG. 2), is obtained. This could include obtaining demographic or personal history information from the patient or others, performing clinical or laboratory measurements (e.g., blood pressure, height, weight), or otherwise generating the patient data 101. Additionally or alternatively, some or all of the patient data 101 could be obtained from a database of such information (e.g., from a patient's medical record as stored in a secure hospital database). In examples wherein the data is obtained from a database of medical records, the method of FIG. 1 could be re-run periodically, e.g., in response to an update to a particular patient's medical record, in order to detect when the patient's risk increases to a level sufficient to motivate performance of a diagnostic or other intervention. The patient data 101 is then applied to the trained predictor 120 to generate a colorectal cancer risk score for the patient. This risk score is then evaluated 130 (e.g., by comparison to a threshold value) to determine whether the patient's predicted risk is sufficient to motivate the performance of a diagnostic 105 (e.g., acquiring and analyzing a stool sample from the patient, performing a colonoscopy on the patient). The results of the diagnostic may then optionally be evaluated 140 and used to identify the patient as being at increased risk of, or having, colorectal cancer and to responsively provide an amount effective of a therapy 107 to treat or limit development of the colorectal cancer.

In some examples, human experience (e.g., the intuition of a medical practitioner) could be injected into the process. For example, responsive to determining 130 that the risk score is high, a clinician or other user could be provided with a display, on a user interface, that indicates that the risk score is high (e.g., by indicating a percentile of the risk score, by displaying the risk score itself, by providing additional information like some or all of the underlying patient data 101). The user could then provide (e.g., via the same user interface) an indication (e.g., a button press or other user interface interaction) that the colorectal cancer diagnostic test should be performed on the patient. Responsive to that input, the diagnostic test 105 could be performed.

The computational cost of the embodiments described herein could be further reduced by selectively applying the predictor 120. This could include first using the patient data 101 to evaluate 110 the patient's colorectal cancer risk based on known ‘conventional’ indicators or heightened colorectal cancer risk. Such ‘conventional’ indicators could include one or more of a personal or familial history of colorectal polyps or colorectal cancer, a personal history of inflammatory bowel disease (e.g., Crohn's, IBS, colitis), or the presence of one of a specified set of inherited syndromes that is correlated with an increased risk of colorectal cancer (e.g., Lynch Syndrome, familial adenomatous polyposis (FAP), attenuated FAP, Gardner syndrome, Turcot syndrome, Peutz-Jeghers syndrome, MUTYH-associated polyposis). As shown in FIG. 1, evaluation 110 of the patient data with respect to one or more of those factors could be performed first. If any of those factors is positive or otherwise indicates an elevated colorectal cancer risk, the diagnostic 105 could be responsively performed. If not, the predictor model 120 could be executed on the patient data 101 to evaluate whether the patient, despite not exhibiting any of the ‘conventional’ risk factors, still exhibits sufficiently elevated risk of colorectal cancer to motivate performance of the diagnostic 105.

The feature selection and model training methods described herein provide a variety of benefits. Among these benefits are the ability to evaluate large number of potential predictive features, many of which may be unexpected or otherwise thought unlikely to be relevant to colorectal cancer risk. For example, the predictor 120 could receive as input features some or all of employment status, a number of medical encounters in the previous year, whether a patient has been screened for colorectal cancer, age, whether a patient has a diagnosis related to the digestive system, how many colonoscopies a patient has received, whether a patient has a diagnosis related to the nervous system, a number of days since a patient's last colonoscopy, whether a patient has been prescribed antidepressants, whether a patient has a diagnosis related to a parasitic infection, whether a patient has been prescribed anti-hyperlipidemia medication, a level of alcohol use, whether a patient has been prescribed a dermatological medication, sex, whether a patient has been prescribed anti-rhematic medication, and marital status. Accordingly, predictions made as described herein can be more accurate and more sensitive due, in part, to the ability to take advantage of the predictive power of such large number of features, including ‘unconventional’ features. Indeed, in some examples, the input feature set to the predictor 120 could intentionally omit the set of ‘conventional’ features above, since those features could be used as part of an initial risk screening phase. Such ‘unconventional’ features could include at least one of marital status or employment status.

As used herein, a “control” may be any suitable control for comparing colorectal risk scores, including but not limited to risk scores from patients that do not have colorectal cancer and/or or not at high risk of colorectal cancer (whether simultaneously measured or historical data). Those of skill in the art will recognize that other such controls can be used in the methods of the disclosure.

In one embodiment, the methods described herein can further include treating a patient identified as being at increased risk of, or having, colorectal cancer, with an amount effective of a therapy to treat or limit development of the colorectal cancer. In this embodiment, the methods provide improved treatment and diagnostic methods all by administering the therapies to those subjects that will most benefit from the administration. As will be understood by those of skill in the art based on the teachings herein, these methods can also be used to monitor therapy for colorectal cancer.

As used herein for all embodiments, “treat” or “treating” means accomplishing one or more of the following: (a) reducing the size or volume of tumors and/or metastases in the subject; (b) limiting any increase in the size or volume of tumors and/or metastases in the subject; (c) increasing survival or progression-free survival; (d) reducing the severity of symptoms associated with cancer; (e) limiting or preventing development of symptoms associated with cancer; and (f) inhibiting worsening of symptoms associated with cancer.

The embodiments described herein were experimentally implemented and assessed. The results of that evaluation, with respect to specific implementations, are described below.

In a first experiment, a machine learning model was developed to predict one's likelihood of a CRC diagnosis. A retrospective dataset of all active Sanford patients (face to face clinical encounter in the last 27 months) was built, containing relevant clinical risk factors from a myriad of EMR data sources: demographics, vitals, lab values, diagnoses, medications, etc., The model was evaluated on 20% of the population of interest and demonstrated high performance (AUC=0.88). Furthermore, the model has been shown to be associated with non-traditional stool-based testing (AUC=0.64) in a prospective cohort of Sanford patients. The model can be used to help clinicians identify patients at higher risk for CRC and to provide to them earlier diagnostic or other interventions.

In a second, later experiment, model development was undertaken using a retrospective cohort of Sanford patients that were clinically active from 2017-2023. The target was a pathology confirmed diagnosis of colorectal cancer; those without a diagnosis were controls. This data curation comprised of 4,644 patients with a diagnosis and 455,235 without. All data were queried via an automated EMR data extraction from Sanford's Clarity database which populates clinical data within Sanford. Data sources of potential predictor variables included: patient demographics, diagnosis history, vitals, medication history, lab values, and screening history. Initial curation of numeric potential predictor variables were aggregated over the study period and nominal, list-like sources (diagnosis history, medication history) were stored in a comma-separated format for clinically relevant binning based on open-source resources. Specifically, pharmacy classes and the Clinical Classification Software (css) were used to identify and engineer features that represent the presence of the presence of sed group.

209 initial candidate variables were initially curated based on theoretical relevance for further statistical analysis and justification to be included in modeling. Both bivariate and multivariate modeling techniques were implemented to identify statistically relevant variables. Traditional hypothesis testing and data visualization were used for bivariate analyses. If no independent association was identified via bivariate analysis (predictor variable and colorectal cancer specification), then it was not considered for modeling. Specifically, bootstrapped estimates of association were calculated with chi-squared tests and Fisher's exact tests for nominal variables and Wilcoxon rank sum tests for numeric variables. Inferences on magnitude and confidence were drawn from visualization and intervals of a potential effect. This filtered the initial 209 variables to approximately 100 variables, which were then subjected to multivariate modeling for feature importance estimation. An initial machine learning model was trained on all variables and SHAP values were calculated to rank order and quantify the direction of how each variable related to a diagnosis of colorectal cancer. SHAP (Shapley Additive Explanations) values are a game-theoretic method of calculating the contribution of each variable to a predicted value. This can be used in machine learning contexts to generate a rank-ordered estimation of importance across all variables. Using these estimates, a final list of 77 variables (See Table 1 below) was selected to be used for modeling

TABLE 1

| _Predictor name | Predictor description |
|---|---|
| sex | assigned at birth |
| age | at time of modeling index |
| race | manually patient specified race |
| employement_status | at time of modeling index |
| marital_status | at time of modeling index |
| has_pcp | 0 = no PCP, 1 = has PCP at time of modeling index |
| ruca1 | Patients level of rurality as specified via RUCA |
| enctrs_last_year | Number of clinical appointments made in last year |
| avg_diastolic | average diastolic blood pressure |
| avg_spo2 | average SPO2 saturation |
| labs_wbc | average white blood cell count |
| labs_hemoglobin | average hemoglobin |
| labs_pltcount | average platelet count |
| labs_mcv | average mean corpuscular volume |
| labs_mch | average mean corpuscular hemoglobin |
| labs_rdwcv | average red cell distribution width -ceofficient of variation |
| labs_rdwsd | average red cell distribution width - standard deviation |
| labs_neutroabs | average absolute neutrophil count |
| labs_cl | average chloride |
| labs_ca | average calcium |
| labs_alkphos | average alkaline phosphatase |
| labs_albumin | average albumin |
| alcohol_use | alcohol use at time of modeling index |
| smoking | smoking status at modeling index |
| med_aminoglycosides | Taking 1+ aminoglyosides medication |
| med_anti_rheumatic | Taking 1+ rhematic medication |
| med_anticoagulants | Taking 1+ anticoagulant |
| med_anticonvulsant | Taking 1+ anticonvulsant |
| med_antidepressants | Taking 1+ antidepressant |
| med_antihyperlipidemic | Taking 1+ antihyperlipidemic |
| med_antiviral | Taking 1+ antiviral |
| med_cough_cold | Taking 1+ cough/cold |
| med_decongestants | Taking 1+ decongestants |
| med_dermatological | Taking 1+ dermatological |
| med_general_anesthetics | Taking 1+ general anesthetics |
| med_misc_gi | Taking 1+ GI |
| med_multivitamins | Taking 1+ multivitamin |
| med_ophthalmmic | Tking 1+ opthalmmic |
| med_skeletal_muscle_relaxants | Taking 1+ skeletal/muscle relaxant |
| med_thyroid | Taking 1+ thyroid |
| med_vitamins | Taking 1+ vitamin |
| dx_diseases_of_the_blood_and_blood_forming_organs | 1+ diagnosis within blood and/or blood forming organs |
| dx_diseases_of_the_circulatory_system | 1+ diagnosis of circulatory system |
| dx_diseases_of_the_digestive_system | 1+ diagnosis of digesitive system |
| dx_diseases_of_the_genitourinary_system | 1+ diagnosis of genitourinary system |
| dx_diseases_of_the_musculoskeletal_{—} | 1+ diagnosis of musculoskeletal system |
| system_and_connective_tissue | |
| dx_diseases_of_the_nervous_system_and_sense_organs | 1+ diagnosis of nervous system |
| dx_diseases_of_the_respiratory_system | 1+ diagnosis of respiratory system |
| dx_diseases_of_the_skin_and_subcutaneous_tissue | 1+ diagnosis of skin/subcutaneous tissue |
| dx_endocrine_nutritional_and_metabolic_{—} | 1+ diagnosis of endorine system and/or |
| diseases_and_immunity_disorders | metabolic/immunity disorders |
| dx_infectious_and_parasitic_diseases | 1+ diagnosis of infectious or parastic nature |
| dx_injury_and_poisoning | 1+ diagnosis of injury/poisoning |
| dx_mental_illness | 1+ diagnosis of mental illness |
| dx_symptoms_signs_and_ill_defined_conditions_{—} | 1+ diagnosis of ill definied nature |
| and_factors_influencing_health_status | influencing patient health |
| dx_inflammatory_bowl_disease | inflammatory bowl disease |
| dx_rectal_bleeding | History of rectal bleeding |
| dx_unexplained_iron_deff | unexplained iron defficiency |
| dx_unexplained_weight_loss | unexplained weight loss |
| dx_change_in_bowel_habbits | Recent change in bowel habbits |
| dx_familial_adenomatous_polyposis | familiar adnomaous polyposis |
| dx_mutyh | MUTYH |
| dx_peurtz_jeghers | Peurtz-Jeghers syndrome |
| dx_anemia | History of anemia |
| dx_gastrointestinal_hemorrhage | History of gastrointestinal hemorrhage |
| dxhx_cancer | Family history of cancer (any) |
| dxhx_diabetes | Family history of diabetes |
| dxhx_depression | Family history of depression |
| colonoscopies | Number of colonoscopies ordered |
| cologaurds | Number of Cologuards ordered |
| fits | Number of FIT tests ofdered |
| sigmoidoscopies | Number of sigmoidosopies orderes |
| days_last_colonoscopy | Number of days since last colonocopy |
| days_last_cologaurd | Number of days since last Cologuard |
| days_last_fit | Number of days since last FIT test |
| days_last_sigmoidoscopy | Number of days since last sigmoidoscopy |
| screened | Ever screened in lifetime, 0 = never, 1 = yes |

The 77 predictor variables and the outcome variable, which represented presence of a colorectal cancer diagnosis, were used to train a machine learning model to estimate risk. To balance model accuracy without overfitting the training data, 80% of the total dataset was used for model training, with 20% reserved for testing. A CatBoost model was selected. CatBoost is a derivation of XGBOOST, which permits modeling of nominal features. XGBOOST is an open-source framework for both classification and regression tasks using gradient-boosted decision trees. K-fold cross validation was used for hyperparameter tuning with 10 folds and 3 repeats. Area under the curve (AUC) was used as the metric of model performance.

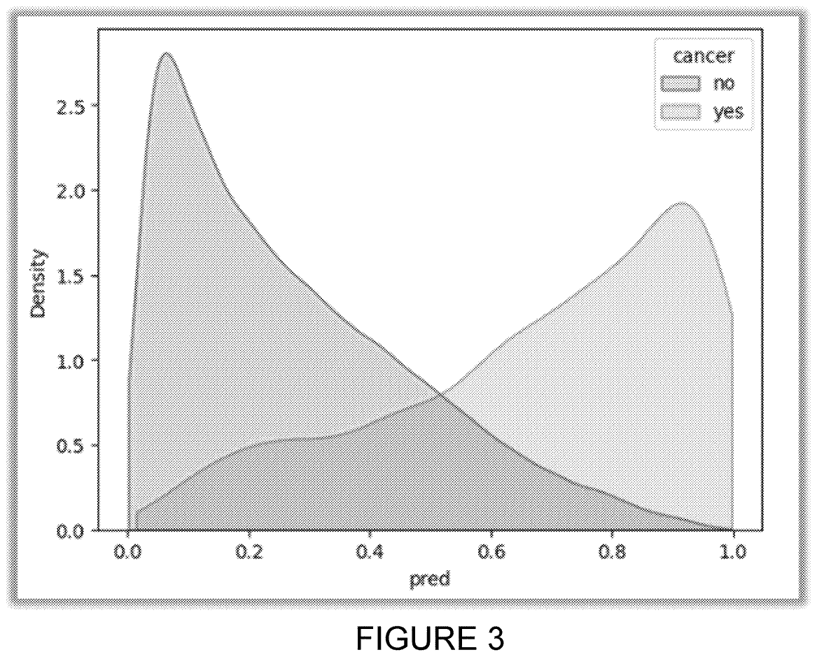

When the finalized model was evaluated on the testing data, it yielded an AUC of 0.88 and demonstrated a large discrimination between those with and without a colorectal cancer diagnosis (FIG. 3). At a risk threshold of a 0.5 (range 0-1), there was a 0.76 sensitivity and 0.84 specificity.

All clinically engaged patients (an in-person clinical visit in the prior 2 years) as of December of 2023 (n=422,182) had their risk calculated for a cohort analysis. Patient metrics were collected over the subsequent 18 months to evaluate how the risk score performed. There were three outcomes of interest: 1) a positive stool-based test, 2) a pre-cancerous polyp identification, and 3) a colon cancer diagnosis.

Cologuard kits were mailed directly to patients to encourage screening and reduce the burden of organizational demand of screening colonoscopy for average risk patients (n=1,733 returned kits in May 2024). Patients were chart reviewed by Sanford clinicians, allowing for more specific clinical outcomes not typically documented reliability within the EHR. Specifically, pre-cancerous polyps were labeled for patients that had colonoscopy ordered for patients with a positive stool-based test. A positive stool-based test (outcome 1) served as an important secondary outcome to the primary outcome used for prospective validation, i.e., a colorectal cancer diagnosis. Chart-reviewed documentation of pre-cancerous polyps served as another secondary outcome (outcome 2) to evaluate the scientific validity and utility of the risk score. Lastly, patients scored in December of 2023 in the full general population (n=422,182) had all potential colorectal cancer diagnoses pulled via an automated data pull to evaluate an association with the risk score.

### Outcome 1

The colorectal cancer risk score associated with a positive test (AUC=0.65). Patients with a score of 70% or more were 3.5× more likely to have a positive test (p<0.001).

### Outcome 2

Patients with a positive stool-based test and a subsequent colonoscopy were chart reviewed for presence of a pre-cancerous polyp. Patients with a precancerous polyp had a significantly elevated risk score compared to those without a precancerous polyp (p<0.05).

### Outcome 3

Over the prospective 18 months in the general population, patients with a score of 70% or higher were 3.4× more likely to have a colorectal cancer diagnosis (p<0.001). This result was found above and beyond the presence of a traditional risk factor, which increased risk at a lesser rate of 1.37× (p<0.001).

In order to estimate generalizability of the model in other populations, a data-sharing collaboration allowed for analyzing ~1 million patients located in San Diego and Texas. The demographic of these datasets was much different from the largely Caucasian sample of Sanford. Similar to Sanford's population, a traditional risk factor was shown to increase a patient's risk of a colorectal cancer diagnosis by 1.5× (p<0.001). Above and beyond a traditional risk factor, flagging the same proportion of patients (10%), the colorectal cancer risk score increased one's risk 4.8× (p<0.001). There were no meaningful differences in model performance between race or ethnicity.

In the experiment, deployment of the colorectal cancer risk score was designed to inform clinicians of patients at a particular threshold of estimated colorectal cancer risk and not to make clinical decisions of any kind. That is, the colorectal cancer risk score was delivered in such a way that clinicians could use it to help deliver the best care they thought appropriate.

Evaluation of patient medical records was performed at a cadence to score all clinically engaged patients each month. Once predictions were generated, messaging was used to send each patient's updated risk score within its EPIC system.

Clinicians were notified of a patient's potential risk based on where they fell on the distribution of the risk score. There were 8 infographics generated for 8 different risk thresholds (50^{th}, 60^{th}, 70^{th}, 80^{th}, 90^{th}, 95^{th}, 97.5^{th}, 99^{th} percentile) and corresponding risk increases. If a patient had a score at or above the 50^{th} percentile of the risk score, an infographic appeared on the patient's EPIC Storyboard-which a clinician could hover over to investigate the information. If the clinician chose, they could click into an EPIC SmartSet to order a colorectal cancer screen (stool-based test, colonoscopy, or other diagnostic) for that particular patient.

A third experiment was performed in order to evaluate the importance of traditional (having a reasonable amount of support within the literature of being relevant, like personal or familial of colorectal polyps or colorectal cancer) vs non-traditional (little/inconsistent/no support within the literature, like marital status or employment status) feature importance, 3 separate models were trained: 1) using all features identified as having a statistical association (traditional and non-traditional) as justified with bivariate hypothesis testing against colon cancer diagnosis, 2) using only traditional risk factors, and 3) using only non-traditional risk factors. Feature importance was compared between these models using SHAP values. Additionally, performance was evaluated as a function of the number of non-traditional features used—quantifying the number of non-traditional features to be included in modeling.

The model with only traditional risk factors (n=32) obtained an AUC of 0.81 while the model with only non-traditional risk factors (n=46) obtained an AUC of 0.78. When every feature was included (n=78) the model reached an improved AUC of 0.88. When predictions were labeled as cancer at or above the 80^{th} percentile of each score, sensitivity increased from 0.65 when only using traditional risk factors to 0.79 when using both traditional and non-traditional risk factors—resulting in a 13.5% improvement in cancer diagnoses detected.

In the overall model, employment status, age, number of prior clinical visits in the last year, whether or not the patient had been screened, and average blood albumin value were the top 5 most important features. Of the top 20 most important features, 9 were traditional risk factors whereas 11 were non-traditional risk factors.

A relatively linear increase was observed with respect to AUC as the number of features increased. When considering nearly all non-traditional risk factors excluded (98%), 50% of non-traditional factors excluded, and nearly all non-traditional factors excluded (2%), there was a clear trend of continued increased importance of these features (AUC=0.81, 0.86, 0.88 respectively).

### II. EXAMPLE METHODS

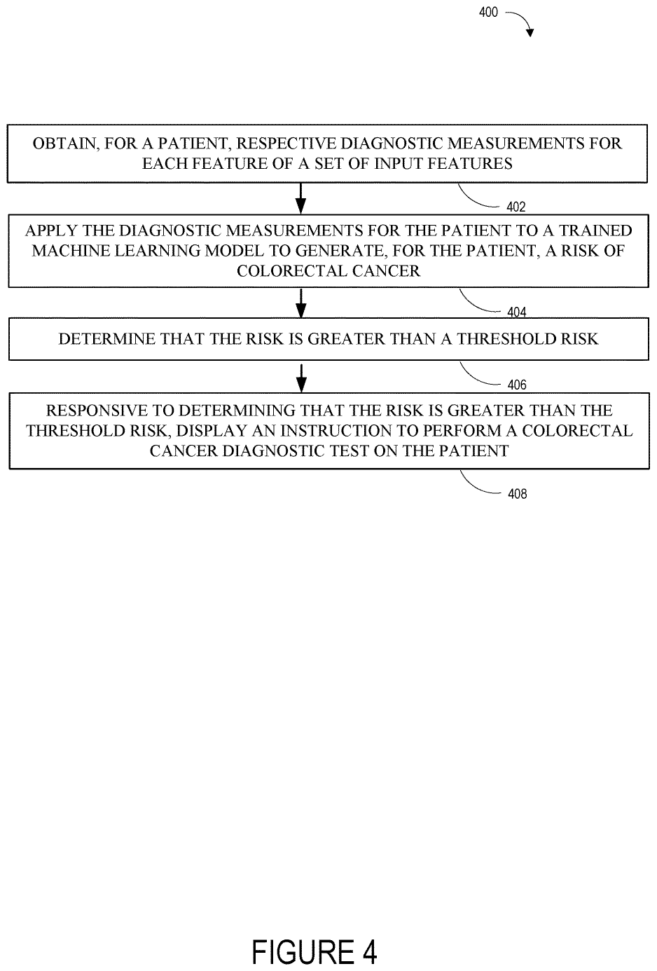

FIG. 4 depicts an example method 400 for diagnosing a patient as at an increased risk of colorectal cancer relative to control. The method 400 includes obtaining, for a patient, respective diagnostic measurements for each feature of a set of input features (402). The method 400 additionally includes applying the diagnostic measurements for the patient to a trained machine learning model to generate, for the patient, a risk of colorectal cancer (404). The trained machine learning model has been trained via a training process that includes: (i) obtaining a training dataset that comprises, for each subject of a plurality of subjects, a corresponding set of diagnostic measurements for each feature of a set of candidate features and a corresponding observed risk of colorectal cancer; (ii) based on the training dataset, determining a first subset of the candidate features that are more predictive of the observed risk of colorectal cancer with respect to a bivariate correlative statistic; (iii) determining a second subset of features of the first subset that are more predictive of the observed risk of colorectal cancer based on training respective predictive models that omit the respective features of the first subset, wherein the second subset of features is the set of input features; and (iv) training the trained machine learning model to predict risk of colorectal cancer using the second subset of features. The method 400 additionally includes determining that the risk is greater than a threshold risk (406). The method 400 additionally includes, responsive to determining that the risk is greater than the threshold risk, displaying an instruction to perform a colorectal cancer diagnostic test on the patient (408). The method 400 could include additional steps or features.

### III. EXAMPLE MACHINE LEARNING MODELS AND TRAINING THEREOF

A machine learning model, trained predictor model, predictor model, or other trained predictive algorithm as described herein may include, but is not limited to: an artificial neural network (e.g., a herein-described convolutional neural networks, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures.

An artificial neural network (ANN) could be configured in a variety of ways. For example, the ANN could include two or more layers, could include units having linear, logarithmic, or otherwise-specified output functions, could include fully or otherwise-connected neurons, could include recurrent and/or feed-forward connections between neurons in different layers, could include filters or other elements to process input information and/or information passing between layers, or could be configured in some other way to facilitate the generation of predicted color palettes based on input images.

An ANN could include one or more filters that could be applied to the input and the outputs of such filters could then be applied to the inputs of one or more neurons of the ANN. For example, such an ANN could be or could include a convolutional neural network (CNN), a Stacked Residual Network, or some other variety of ANN. Convolutional neural networks are a variety of ANNs that are configured to facilitate ANN-based classification or other processing based on medical images, medical records, or other large-dimensional inputs whose elements are organized within two or more dimensions. The organization of the ANN along these dimensions may be related to some structure in the input structure (e.g., as relative location within the two-dimensional space of an image can be related to similarity between pixels of the image).

In example embodiments, a CNN includes at least one one-dimensional (or higher-dimensional) filter that is applied to an input; the filtered input is then applied to neurons of the CNN (e.g., of a convolutional layer of the CNN). A set of neurons of a CNN could receive respective inputs that are determined by applying the same filter to an input. Additionally or alternatively, a set of neurons of a CNN could be associated with respective different filters and could receive respective inputs that are determined by applying the respective filter to the input. Such filters could be trained during training of the CNN or could be pre-specified. For example, such filters could represent wavelet filters, center-surround filters, or some other pre-specified filter patterns.

A CNN or other variety of ANN could include multiple convolutional layers (e.g., corresponding to respective different filters and/or features), pooling layers, rectification layers, fully connected layers, or other types of layers. Convolutional layers of a CNN represent convolution of an input with a filter. Pooling layers of a CNN apply non-linear downsampling to higher layers of the CNN, e.g., by applying a maximum, average, L2-norm, or other pooling function to a subset of neurons, outputs, or other features of the higher layer(s) of the CNN. Rectification layers of a CNN apply a rectifying nonlinear function (e.g., a non-saturating activation function, a sigmoid function) to outputs of a higher layer. Fully connected layers of a CNN receive inputs from many or all of the neurons in one or more higher layers of the CNN. The outputs of neurons of one or more fully connected layers (e.g., a final layer of an ANN or CNN) could be used to determine information about portions of an input or for the input as a whole.

Neurons in a CNN can be organized according to corresponding dimensions of the input. Connections between neurons and/or filters in different layers of the CNN could be related to such times. For example, a neuron in a convolutional layer of the CNN could receive an input that is based on a convolution of a filter with a portion of the input, or with a portion of some other layer of the CNN, that is at a time proximate to the location of the convolutional-layer neuron. In another example, a neuron in a pooling layer of the CNN could receive inputs from neurons, in a layer higher than the pooling layer (e.g., in a convolutional layer, in a higher pooling layer), that have locations that are proximate to the location of the pooling-layer neuron.

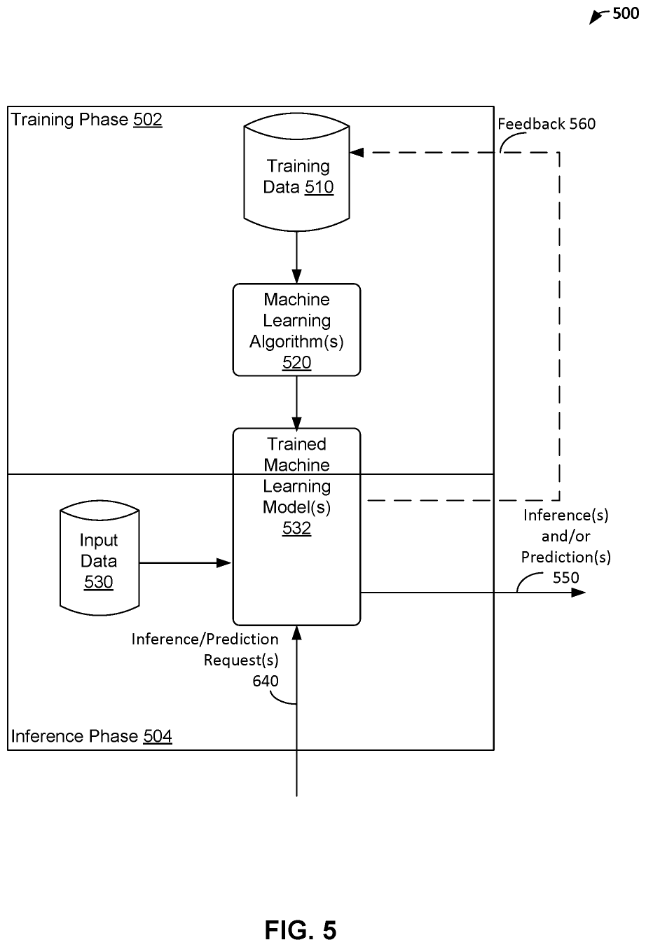

FIG. 5 shows diagram 500 illustrating a training phase 502 and an inference phase 504 of trained machine learning model(s) 532, in accordance with example embodiments. Some machine learning techniques involve training one or more machine learning algorithms, on an input set of training data to recognize patterns in the training data and provide output inferences and/or predictions about (patterns in the) training data. The resulting trained machine learning algorithm can be termed as a trained machine learning model. For example, FIG. 5 shows training phase 502 where one or more machine learning algorithms 520 are being trained on training data 510 to become trained machine learning model 532. Then, during inference phase 504, trained machine learning model 532 can receive input data 530 and one or more inference/prediction requests 540 (perhaps as part of input data 530) and responsively provide as an output one or more inferences and/or predictions 550.

As such, trained machine learning model(s) 532 can include one or more models of one or more machine learning algorithms 520. Machine learning algorithm(s) 520 may include, but are not limited to: an artificial neural network (e.g., a herein-described convolutional neural networks, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures. Machine learning algorithm(s) 520 may be supervised or unsupervised, and may implement any suitable combination of online and offline learning.

In some examples, machine learning algorithm(s) 520 and/or trained machine learning model(s) 532 can be accelerated using on-device coprocessors, such as graphic processing units (GPUs), tensor processing units (TPUs), digital signal processors (DSPs), and/or application specific integrated circuits (ASICs). Such on-device coprocessors can be used to speed up machine learning algorithm(s) 520 and/or trained machine learning model(s) 532. In some examples, trained machine learning model(s) 532 can be trained, reside and execute to provide inferences on a particular computing device, and/or otherwise can make inferences for the particular computing device.

During training phase 502, machine learning algorithm(s) 520 can be trained by providing at least training data 510 as training input using unsupervised, supervised, semi-supervised, and/or reinforcement learning techniques. Unsupervised learning involves providing a portion (or all) of training data 510 to machine learning algorithm(s) 520 and machine learning algorithm(s) 520 determining one or more output inferences based on the provided portion (or all) of training data 510. Supervised learning involves providing a portion of training data 510 to machine learning algorithm(s) 520, with machine learning algorithm(s) 520 determining one or more output inferences based on the provided portion of training data 510, and the output inference(s) are either accepted or corrected based on correct results associated with training data 510. In some examples, supervised learning of machine learning algorithm(s) 520 can be governed by a set of rules and/or a set of labels for the training input, and the set of rules and/or set of labels may be used to correct inferences of machine learning algorithm(s) 520.

Once training phase 502 has been completed, trained machine learning model(s) 532 can be provided to a computing device, if not already on the computing device. Inference phase 504 can begin after trained machine learning model(s) 532 are provided to a computing device.

During inference phase 504, trained machine learning model(s) 532 can receive input data 530 and generate and output one or more corresponding inferences and/or predictions 550 about input data 530. As such, input data 530 can be used as an input to trained machine learning model(s) 532 for providing corresponding inference(s) and/or prediction(s) 550 to kernel components and non-kernel components. For example, trained machine learning model(s) 532 can generate inference(s) and/or prediction(s) 550 in response to one or more inference/prediction requests 540. In some examples, trained machine learning model(s) 532 can be executed by a portion of other software. For example, trained machine learning model(s) 532 can be executed by an inference or prediction daemon to be readily available to provide inferences and/or predictions upon request. Input data 530 can include data from a computing device executing trained machine learning model(s) 532 and/or input data from one or more computing devices other than the executing device.

### IV. ILLUSTRATIVE SYSTEMS

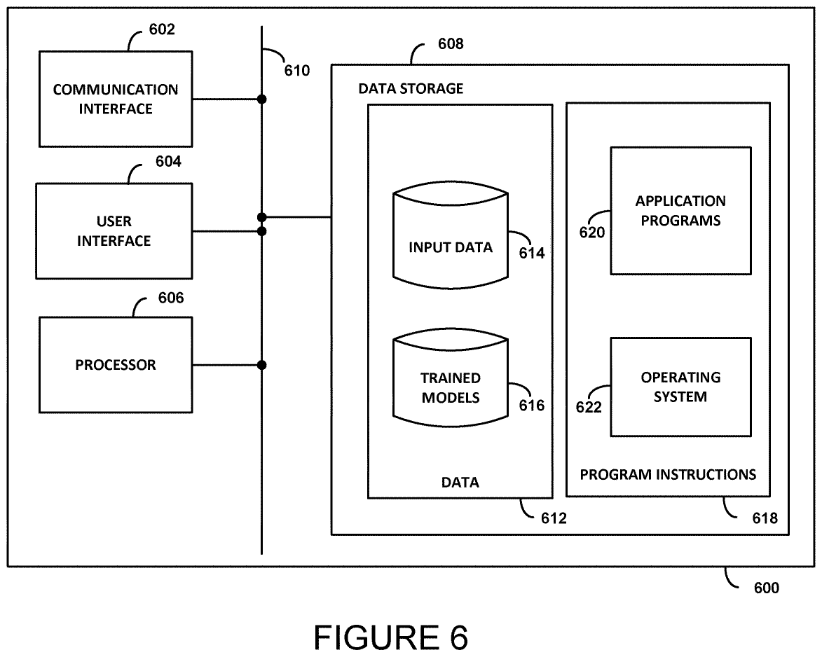

FIG. 6 illustrates an example computing device 600 that may be used to implement the methods described herein. By way of example and without limitation, computing device 600 may be a cellular mobile telephone (e.g., a smartphone), a computer (such as a desktop, notebook, tablet, or handheld computer, a server), elements of a cloud computing system, or some other type of device. It should be understood that computing device 600 may represent a physical computing device such as a server, a particular physical hardware platform on which a prediction and/or scheduling application operates in software, or other combinations of hardware and software that are configured to carry out functions as described herein.

As shown in FIG. 6, computing device 600 may include a communication interface 602, a user interface 604, a processor 606, and data storage 608, all of which may be communicatively linked together by a system bus, network, or other connection mechanism 310.

Communication interface 602 may function to allow computing device 600 to communicate, using analog or digital modulation of electric, magnetic, electromagnetic, optical, or other signals, with other devices, access networks, and/or transport networks. Thus, communication interface 602 may facilitate circuit-switched and/or packet-switched communication, such as plain old telephone service (POTS) communication and/or Internet protocol (IP) or other packetized communication. For instance, communication interface 602 may include a chipset and antenna arranged for wireless communication with a radio access network or an access point. Also, communication interface 602 may take the form of or include a wireline interface, such as an Ethernet, Universal Serial Bus (USB), or High-Definition Multimedia Interface (HDMI) port. Communication interface 602 may also take the form of or include a wireless interface, such as a Wi-Fi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 602. Furthermore, communication interface 602 may comprise multiple physical communication interfaces (e.g., a Wi-Fi interface, a BLUETOOTH® interface, and a wide-area wireless interface).

In some embodiments, communication interface 602 may function to allow computing device 600 to communicate, with other devices, remote servers, access networks, and/or transport networks. For example, the communication interface 602 may function to access one or more machine learning models or other predictive algorithms and/or input therefor via communication with a remote server or other remote device or system in order to allow the computing device 600 to use the machine learning model or algorithms to generate outputs (e.g., predicted risk scores with respect to the future or current occurrence of colorectal cancer) based on input data. For example, the computing system 600 could be a medical records server and the remote system could be a smartphone via which a medical care provider interacts to determine whether to provide a patient with stool sample screening, a colonoscopy, or some other elevated diagnostic method.

User interface 604 may function to allow computing device 600 to interact with a user, for example to receive input from and/or to provide output to the user. Thus, user interface 604 may include input components such as a keypad, keyboard, touch-sensitive or presence-sensitive panel, computer mouse, trackball, joystick, microphone, and so on. User interface 604 may also include one or more output components such as a display screen which, for example, may be combined with a presence-sensitive panel. The display screen may be based on CRT, LCD, and/or LED technologies, or other technologies now known or later developed. User interface 604 may also be configured to generate audible output(s), via a speaker, speaker jack, audio output port, audio output device, earphones, and/or other similar devices.

Processor 606 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). In some instances, special purpose processors may be capable of executing artificial neural networks, executing convolutional neural networks, or executing some other predictive models or algorithms among other applications or functions. Data storage 608 may include one or more volatile and/or non-volatile storage components, such as magnetic, optical, flash, or organic storage, and may be integrated in whole or in part with processor 606. Data storage 608 may include removable and/or non-removable components.

Processor 606 may be capable of executing program instructions 618 (e.g., compiled or non-compiled program logic and/or machine code) stored in data storage 608 to carry out the various functions described herein. Therefore, data storage 608 may include a non-transitory computer-readable medium, having stored thereon program instructions that, upon execution by computing device 600, cause computing device 600 to carry out any of the methods, processes, or functions disclosed in this specification and/or the accompanying drawings. The execution of program instructions 618 by processor 606 may result in processor 606 using data 612.

By way of example, program instructions 618 may include an operating system 622 (e.g., an operating system kernel, device driver(s), and/or other modules) and one or more application programs 620 (e.g., functions for executing trained machine learning models and/or other predictive methods as described herein) installed on computing device 600. Data 612 may include medical records data or some other input 614 and/or one or more trained machine learning models 616 or other elements of a predictive method as described herein. Input data 614 may be used to train predictors (e.g., machine learning models thereof) and/or may be applied to such a predictor in order to generate a likelihood of colorectal cancer or some other medical condition, to determine whether to perform a stool sample collection, colonoscopy, or other diagnostic method on a patient, or to generate some other output as described herein.

Application programs 620 may communicate with operating system 622 through one or more application programming interfaces (APIs). These APIs may facilitate, for instance, application programs 620 reading and/or writing a trained predictive model 616, transmitting or receiving information via communication interface 602, receiving and/or displaying information on user interface 604, and so on.

Application programs 620 may take the form of “apps” that could be downloadable to computing device 600 through one or more online application stores or application markets (via, e.g., the communication interface 602). However, application programs can also be installed on computing device 300 in other ways, such as via a web browser or through a physical interface (e.g., a USB port) of the computing device 600.

### VI. CONCLUSION

The above detailed description describes various features and functions of the disclosed systems, devices, and methods with reference to the accompanying figures. In the figures, similar symbols typically identify similar components, unless the context indicates otherwise. The illustrative embodiments described in the detailed description, figures, and claims are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flowcharts in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random access memory (RAM). The computer-readable media may also include non-transitory computer-readable media that stores program code and/or data for longer periods of time, such as secondary or persistent long term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

We claim:

1. A method for diagnosing a patient as at an increased risk of colorectal cancer relative to control comprising:
obtaining, for a patient, respective diagnostic measurements for each feature of a set of input features;
applying the diagnostic measurements for the patient to a trained machine learning model to generate, for the patient, a risk of colorectal cancer, wherein the trained machine learning model has been trained via a training process that comprises:
obtaining a training dataset that comprises, for each subject of a plurality of subjects, a corresponding set of diagnostic measurements for each feature of a set of candidate features and a corresponding observed risk of colorectal cancer;
based on the training dataset, determining a first subset of the candidate features that are more predictive of the observed risk of colorectal cancer with respect to a bivariate correlative statistic;
determining a second subset of features of the first subset that are more predictive of the observed risk of colorectal cancer based on training respective predictive models that omit the respective features of the first subset, wherein the second subset of features is the set of input features; and
training the trained machine learning model to predict risk of colorectal cancer using the second subset of features;
determining that the risk is greater than a threshold risk; and
responsive to determining that the risk is greater than the threshold risk, displaying an instruction to perform a colorectal cancer diagnostic test on the patient.

2. The method of claim 1, further comprising, responsive to displaying the instruction, performing a colonoscopy on the patient.

3. The method of claim 1, further comprising, responsive to displaying the instruction, obtaining a stool sample from the patient and testing the stool sample.

4. The method of claim 1, further comprising:
based on a performance of the instructed colorectal cancer diagnostic test, identifying the patient as at increased risk of, or having, colorectal cancer; and
responsively treating the patient identified as being at increased risk of, or having, colorectal cancer, with an amount effective of a therapy to treat or limit development of the colorectal cancer.

5. The method of claim 1, wherein displaying the instruction to perform the colorectal cancer diagnostic test on the patient responsive to determining that the risk is greater than the threshold risk comprises:
responsive to determining that the risk is greater than a threshold risk, displaying, via a user interface to a user, an indication of the risk;
receiving, from the user interface, an indication that the colorectal cancer diagnostic test should be performed on the patient; and
responsively displaying the instruction to perform the colorectal cancer diagnostic test on the patient.

6. The method of claim 1, wherein the bivariate correlative statistic includes at least one of a chi-squared test, a Fisher's exact test, or a Wilcoxon rank sum test.

7. The method of claim 1, wherein determining the second subset of features that are based on training respective predictive models that omit the respective features of the first subset comprises:
determining, for each feature in the first subset, a respective Shapley additive explanation (SHAP) score;
ranking the features of the first set according to the SHAP scores; and
selection a subset of the features of the first set that are highest ranked with respect to the ranked SHAP scores.

8. The method of claim 1, wherein the trained machine learning model is an ensemble of weaker models trained via gradient boosting.

9. The method of claim 1, wherein applying the diagnostic measurements for the patient to the trained machine learning model is performed responsive to determining, based on diagnostic measurements for each feature of a set of conventional colorectal predictive features, that the patient is not at risk of colorectal cancer.

10. The method of claim 9, wherein the conventional features comprise at least one of (i) a personal history of colorectal polyps or colorectal cancer, (ii) a family history of colorectal polyps or colorectal cancer, (iii) a personal history of inflammatory bowel disease, or (iv) the patient having a specified inherited syndrome that is correlated with an increased risk of colorectal cancer.

11. The method of claim 9, further comprising:
determining, based on diagnostic measurements for each feature of the set of conventional colorectal predictive features for a second patient, that the second patient is at risk of colorectal cancer; and
responsively, without applying diagnostic measurements for the second patient to the trained machine learning model, performing the colorectal cancer diagnostic test on the second patient.

12. The method of claim 9, wherein the set of input features does not include any of the conventional features.

13. The method of claim 1, further comprising:
obtaining, for a second patient, respective second diagnostic measurements for each feature of the set of input features;
applying the second diagnostic measurements for the second patient to the trained machine learning model to generate, for the second patient, a second risk of colorectal cancer;
determining that the second risk is not greater than the threshold risk; and
responsive to determining that the second risk is not greater than the threshold risk, displaying an instruction not to perform the colorectal cancer diagnostic test on the second patient.

14. The method of claim 1, wherein obtaining, for the patient, respective diagnostic measurements for each feature of the set of input features comprises obtaining the diagnostic measurements from a medical record of the patient that is stored in a database of medical records.

15. The method of claim 14, further comprising:
obtaining, for a second patient, respective second diagnostic measurements for each feature of the set of input features from a second medical record of the second patient that is stored in the database;
applying the second diagnostic measurements for the second patient to the trained machine learning model to generate, for the second patient, a second risk of colorectal cancer;
determining that the second risk is not greater than the threshold risk;
responsive to determining that the risk is not greater than a threshold risk, displaying an instruction not to perform the colorectal cancer diagnostic test on the second patient;
subsequently updating the second medical record based on a measured update to at least one of the second diagnostic measurements;
applying the second diagnostic measurements from the updated second medical record to the trained machine learning model to generate, for the second patient, a third risk of colorectal cancer;
determining that the third risk is greater than the threshold risk; and
responsive to determining that the third risk is greater than the threshold risk, displaying an instruction to perform the colorectal cancer diagnostic test on the second patient.

16. The method of claim 1, wherein the features include employment status, a number of medical encounters in the previous year, whether a patient has been screened for colorectal cancer, age, whether a patient has a diagnosis related to the digestive system, how many colonoscopies a patient has received, whether a patient has a diagnosis related to the nervous system, a number of days since a patient's last colonoscopy, whether a patient has been prescribed antidepressants, whether a patient has a diagnosis related to a parasitic infection, whether a patient has been prescribed anti-hyperlipidemia medication, a level of alcohol use, whether a patient has been prescribed a dermatological medication, sex, whether a patient has been prescribed anti-rhematic medication, and marital status.

17. The method of claim 1, wherein the features include at least one of marital status or employment status.

18. An article of manufacture including a non-transitory computer-readable medium, having stored thereon program instructions that, upon execution by a computing device, cause the computing device to perform the method of claim 1.

19. The article of manufacture of claim 18, wherein the features include at least one of marital status or employment status.

20. The article of manufacture of claim 18, wherein applying the diagnostic measurements for the patient to the trained machine learning model is performed responsive to determining, based on diagnostic measurements for each feature of a set of conventional colorectal predictive features, that the patient is not at risk of colorectal cancer.
